(19) United States
(12) Patent Application Publication
(10) Pub. No.: US 2022/0000788 A1
(43) Pub. Date: Jan. 6, 2022

(54) METHOD FOR DRY POWDER COATING CAPSULES

(71) Applicant: NINGBO WESTON POWDER PHARMA COATING CO., LTD., Ningbo (CN)

(72) Inventors: Jingxu Zhu, LONDON (CA); Yingliang Ma, London (CA); Qingliang Yang, London (CA); Kwok Yui Chow, Mississauga (CA); Herman C. Lam, Scarborough (CA)

(21) Appl. No.: 17/476,716

(22) Filed: Sep. 16, 2021

Related U.S. Application Data
(63) Continuation of application No. 15/722,529, filed on Oct. 2, 2017, now abandoned.

(51) Int. Cl. A61K 9/48; A61K 47/32; A61K 47/36; A61K 47/69

(52) U.S. Cl. CPC A61K 9/4816; A61K 47/32; A61K 9/4866; A61K 47/6943; A61K 9/4891; A61K 47/36

(57) ABSTRACT

The present disclosure provides a process of producing a dry powder coated pharmaceutical capsule. The method includes preparing a dry powder film forming polymer coating composition to be coated onto an outer surface of the capsules, a size of the particulate coating powder being in a range from about 1 nm to about 500 μm. The capsules are placed into an interior of a rotatable housing and may be preheated. The dry powder coating composition is sprayed into the interior of the housing while the rotatable housing is rotating to produce a uniform coating of the dry powder coating composition on the outer surface of the capsules. The coated capsules are cured form a substantially uniform cured.

### FIELD

The present disclosure provides compositions for powder coated capsules and a method for the manufacture of the powder coated capsules.

### BACKGROUND

A capsule is a pharmaceutical dosage form, encapsulating active ingredients (medicines) in a relatively stable shell. Capsules can be divided in two main categories: hard capsules (comprised of two pieces) and soft capsules (comprised of one piece). Hard-shelled capsules are normally used to encapsulate solid forms such as powder, pellets or small tablets. Soft-shelled capsules are mainly used for enclosing semi-solid or liquid forms, perfectly suitable for water insoluble active ingredients.

Compared with other dosage forms such as tablets and pellets, capsule has its unique properties. Medicines or active pharmaceutical ingredients (APIs) can be enclosed in a capsule to mask flavors or unpleasant smells, to reduce contamination of the product, and to protect the active ingredients against oxidation. Capsules are better dosage forms than tablets for drugs with low compressibility and slow dissolution. As compared to tablets, capsules require less adjuncts or excipients. The shells of capsules are physiologically inert and easily and quickly digested in the gastrointestinal tract.

Contrary to coating of pellets and tablets, coating of capsules is independent of the capsule content (APIs and excipients). There are clear advantages resulting from coating a capsule, protecting APIs from damage by the coating process. Also it is much easier and more economy to coat a capsule filling with uncoated particles, granules, pellets or small tablets containing API(s) than coating those particles, granules, pellets or small tablets containing API(s) first and then filling them into a capsule. Prior arts have been developed to produce improved coating capsules. In particular, several earlier attempts have been made to produce stable enteric coated capsules which resist dissolution in the acid stomach and dissolve or disintegrate primarily in the intestines, administrating those drugs causing nausea or gastric distress, or unstable in the acid environment of the stomach. Coating soft capsules are also useful to administrate liquid medications which may be distasteful to the patient.

As a result of toxicity and environmental concerns caused by organic solvent coating, aqueous coating started to dominate in 1990s and remains the preferred approach in the present pharmaceutical industry. Most of the prior arts of capsule coating focused on the aqueous coating process. However, since most of the capsules are made of gelatin, the aqueous coating process of capsules is very sensitive and requires a very long process time due to the aqueous solubility of gelatin substrate, resulting in high costs. A pre-coating can reduce interactions between the gelatin and the aqueous coating solutions or dispersions but is time consuming with complicated processing.

Accordingly, it would be very advantageous to provide a method for dry powder coating capsules without using any organic solvent or water.

### SUMMARY

The present disclosure provides a method of dry powder coating of capsules, including capsule compositions and the coating, using dry powder coating technology, preferably electrostatic powder coating technology, for oral administration. The purposes of the present disclosure is to provide oral pharmaceutical or nutraceutical products of film coated capsules.

In an embodiment there is provided a process of producing a dry powder coated pharmaceutical capsule, comprising:

a) preparing a dry powder film forming polymer coating composition, comprised of particles, to be coated onto an outer surface of the capsules, a size of the particles being in a range from about 1 nm to about 500 μm;

b) placing capsules into an interior of a rotatable housing of a coater and preheating the capsules;

c) spraying the dry powder film forming polymer coating composition into the interior to coat an outer surface of the capsules;

d) rotating the rotatable housing to produce a uniform coating of the dry powder film forming polymer coating composition on the outer surface of the capsules; and

e) curing the dry coated capsules to form a substantially uniform cured film enveloping each capsule.

The capsules may be preheated to a temperature close to a glass transition temperature (Tg) of the polymer(s) contained in the film forming polymer coating composition, wherein the polymers are selected to have a glass transition temperature in a range from about 20 to about 200° C.

The glass transition temperature is in a range from about from 30 to about 100° C.

The glass transition temperature is in a range from about from about 40 to about 60° C.

The method may include spraying a suitable amount of plasticizer into the housing to comingle with the dry powder film forming polymer coating composition. The plasticizer may sprayed into the housing prior to spraying the dry powder film forming polymer coating composition, or it may be sprayed into the housing at the same time with spraying the dry powder film forming polymer coating composition.

The plasticizer may be any one or combination of a liquid pure plasticizer, a plasticizer in a solution, and a dry powder plasticizer.

During curing in the housing the coated capsules may be cured at a temperature in a range from about 30 to about 100° C., and wherein a curing time is up to about 4 hours.

The dry powder film forming polymer coating composition may comprise any polymers that could provide flavoring or taste modifying/masking or moisture barrier include, but not limited to, methylcellulose, hydroxyethyl cellulose, hydroxypropyl cellulose (HPC), hydroxylpropyl methyl cellulose (HPMC) and so on to give a few non-limiting examples.

The dry powder film forming polymer coating composition may comprise water soluble polymers that achieve instant or immediate drug release, comprising, but not limited to, methylcellulose, hydroxyethyl cellulose, hydroxypropyl cellulose (HPC), hydroxylpropyl methyl cellulose (HPMC), and poly(vinylpyrrolidinone) (PVP), polyethylene glycols such as but not limited to PVP, PEG 400, PEG 600, PEG 3350, propylene glycol, polaxamer and povidone, or any combinations of any thereof.

The dry powder film forming polymer coating composition may comprise water insoluble polymers that achieve sustained or controlled drug release, comprising, but not limited to, cellulose acetate, ethylcellulose and cellulose derivatives such as cellulose nitrate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, Eudragit® RL, Eudragit® RS, or any combination of any thereof.

The dry powder film forming polymer coating composition may comprise pH dependent polymers that is insoluble in aqueous medium at pH lower than 5.5 that achieve enteric coating for delayed drug release, including, but not limited to, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, Eudragit® L30D, Eudragit® L100, Eudragit® FS30D, Eudragit® S I00, Hydroxypropylmethylcellulose Acetate Succinate, or any combinations of any thereof.

The dry powder film forming polymer coating composition may comprise plasticizers, anti-tacky agents, pore forming agents or other additives, or any combination of any thereof.

The shell of the capsules may be made of gelatin, or HMPC or any other materials, or any combination of any thereof.

The capsules will contain at least one active agent. The active agent can be in any suitable form. For example, it can be in the form of a powder, pellet, or a granule (Le., an aggregate of smaller units of active agent), or small tablets or any combination of any thereof, coated or uncoated.

One or more drug delivery orifices may be formed through the coating film of the capsules at a position of each of the one or more orifices to form an osmotic capsule. The one or more orifices may be produced by using any one of mechanical drilling, and laser drilling, and an indentation method. One or more drug delivery orifices may also be formed through the coating film and the shell of the capsules at a position of each of the one or more orifices to form an osmotic capsule.

A further understanding of the functional and advantageous aspects of the present disclosure can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1 shows a schematic diagram of the method or process of powder coating capsules according to the present disclosure.

FIG. 2 displays a dissolution profile of aspirin from commercial aspirin tablets and powder coated capsules of Example 1 with Acryl-EZE®; (81 mg; 0-2 hours: pH 1; 2-4 hours: pH 6.8.).

FIG. 3 displays a dissolution profile of aspirin from powder coated capsules of EXAMPLE 1 with Acryl-EZE® after storage under accelerated condition (40° C. in the presence of desiccants) for up to 3 months. 0-2 hours: pH 1. 2-4 hours: pH 6.8.

FIG. 4 displays a dissolution profile of aspirin from powder coated capsules of Example 2 with Eudragit® RS and Eudragit® RL (Coating level 8.5%; pH 7.2)

### DETAILED DESCRIPTION

Various embodiments and aspects of the disclosure will be described with reference to details discussed below. The following description and drawings are illustrative of the disclosure and are not to be construed as limiting the disclosure. The drawings are not to scale. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions.

As used herein the terms “capsule” refers to a range of dosage forms used to enclose active ingredients in a relatively stable shell, allowing them to, for example, be taken orally or be used as suppositories.

The terms “active ingredient” and/or “active agent” refer to active pharmaceutical ingredients (APIs) or drugs.

The phrases “film forming coating powder composition” and/or “film forming polymer powder” refers to the mixture of powders being used to form the coating on the particles and can optionally include other constituents or materials.

The phrase “pore forming agent” refers to the powdered polymers, or liquid polymers, or polymer solutions with small molecular weight that can be used as the pore forming agent in the pharmaceutical coating process. Pore forming agents are water soluble materials which can be sprayed together with coating powders including film forming materials in the powder coating process. After being cured, they would be part of the coating film. After being swallowed and upon contacting with GI tract, those pore forming agents are dissolved and leached out, leaving lots of small holes (micropores) on the film, hence the coating film becomes permeable allowing fluids to move into and dissolve the capsule thereby releasing the active drug or agent.

The phrase “osmotic capsule” refers to a drug delivery system comprised of a capsule containing drug(s) and osmotic agent(s) surrounded by a porous outer film or coat to give a plurality of micropore delivery orifice(s). As the osmotic capsule passes through the gastrointestinal tract (GIT), water is absorbed through the coating film via osmosis, dissolving the capsule shell (Gelatin or HPMC or any other materials) and the resulting osmotic pressure is used to push the active drug through the orifice(s) and/or the coating film. Osmotic capsules have gained tremendous attention owing to its distinct characteristics such as zero order drug release kinetics and drug release independent of pH, food and GIT motility.

The phrase “drug delivery orifice” refers to an orifice with a typical diameter of between about 50 μm to about 1 mm located on the coating film which can be created by many techniques, including but not limited to, mechanical drilling or a laser drilling or indentation method or any other methods.

The phrase “micropores” refers to the pores located on the coating film formed by the pore forming agent during coating process, ranged from 1 nm to 100 μm, preferably from 10 nm to 10 μm, more preferably from 50 nm to 5 μm.

The term “curing” refers to applying an energy source, examples being a heat source such as a heater or an infrared source, or an energy source such as an ultraviolet source, to increase the temperature of the coated particles, so as to solidify or partially solidify a powder coating applied to the surface of the pellets. This heat source can be a hot air flowing through the drum, or a heating element inside the housing but close enough to be able to transfer heat to the drum.

The term “powder coating” refers to a method process to coat particles with film forming powder composition, in other words it refers to a method of forming a film coating around a substrate. The “powder coating” also refers to the particle product coated with film forming polymer powder composition.

Eudragit® is a trade mark of Evonik and Acryl-EZE® is a trade mark of Colorcon.

The present disclosure provides an apparatus and a method of using the apparatus for powder coating pharmaceutical capsules.

After the preparation of capsule a powder coating process us used to obtain the out layer coating of the capsules wherein the coating material contains one or more of film formation polymers, flavoring agents, taste modifying agents, taste masking agents, pH sensitive coating materials, moisture barrier coating materials or a combination thereof.

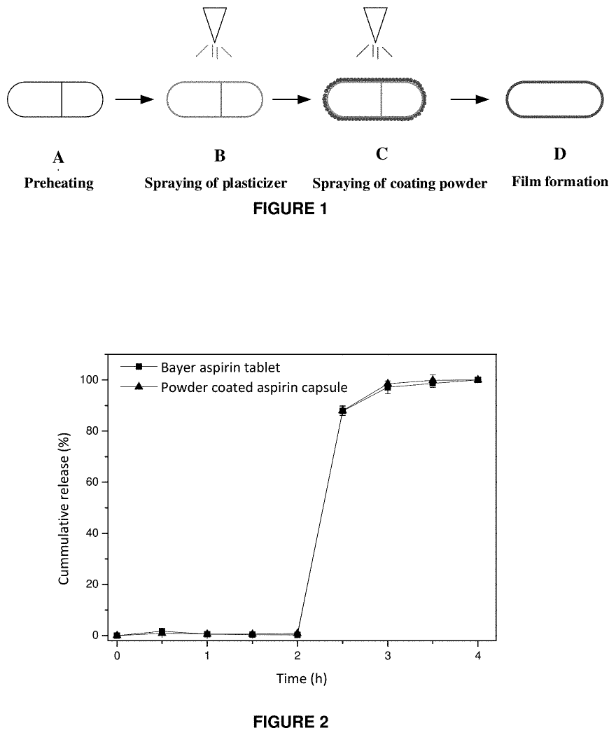

The powder coating process, particularly electrostatic powder coating process comprises the following steps in which steps A to D are schematically illustrated in FIG. 1.
A) Preparation of the powdered coating material is the first step, and in an embodiment the coating powder may be milled using a suitable mill such as an airjet mill, grinder ball mill, pin mill, hammering mill or combination thereof to give particles in a preselected size range. The particle size of coating powder can be in a range of about 1 nm to about 200 μm, preferably in a range of about 10 to about 100 μm, and more preferably in a range from about 20 to about 40 μm. After particle size reduction, those coating materials are mixed together to form a coating formulation.
B) Positioning and preheating is accomplished by loading the capsules into a rotatable housing which has been preheated to a temperature close to the glass transition temperature (T_{g}) of the coating polymers, which is typically in a range from about 30 to about 100° C., preferably from about 30 to about 80° C., more preferably from about 40 to about 60° C.
C) During coating powder deposition the adhesion of the coating powders may need the assistance of a suitable amount of dry powdered plasticizer, or liquid plasticizer or plasticizer solution with a weight ratio range of 0% to about 200% based on weight of the film forming coating powders, preferably in a range from about 5% to about 100%, more preferably in a range from about 10% to about 80%, and in particular preferably in a range of about 20% to about 60%. Plasticizer(s), when present, and film forming coating powders are sprayed onto the surface of the capsules using an air atomizing or airless spray nozzle/electrostatic spray gun (e.g. corona charging gun or a tribo charging gun). If corona gun is used, the voltage can be in a range of about 20 to about 120 kV, preferably in a range of about 25 to about 70 kV, more preferably in a range of about 40 to about 70 kV, and in particular preferably in a range of about 50 to about 70 kV. The plasticizer and coating powders may be sprayed either simultaneously, or via the alternating spray method wherein the plasticizer or powered polymer material is sprayed first and then the other is sprayed and the process may be repeated.

Alternatively, plasticizer can be mixed with powdered material and then this mixture can be sprayed onto the capsule. In all cases, heating preferably continues during the spraying of plasticizer and powdered materials.
D) After the deposition of coating powders, capsules remains in the rotatable housing under a curing temperature, which is in a range from about 30 to about 100° C., preferably from 30 to 80° C., more preferably from about 40 to about 60° C., for a period of time ranged from 0 to about 10 hours, preferably from about 0 to about 4 hours, more preferably from about 1 to about 2 hours, to allow those deposited coating powders to coalesce and form the coating film.

Drug delivery orifice(s) may be drilled on the coating film of the capsules by laser drilling, or mechanical drilling or indentation or any other methods to form osmotic capsules.

The capsule will contain at least one active agent. Typical pharmaceutically active agents include, but are not limited to, e.g., anti-inflammatory, antipyretic, anticonvulsant and/or analgesic agents such as indomethacin, diclofenac, diclofenac Na, ibuprofen, and anti-asthma drugs such as salbutamol and so on. Other APIs having the same or different physiological activity as those above, or suitable mixture thereof, can also be employed in this invention. As used herein, the term “active agent” includes all pharmaceutically acceptable forms of the active agent being described. For example, the active agent can be in an isomeric mixture, a solid complex bound to an ion exchange resin, or the like. In addition, the active agent can be in a solvated form.

The active agent can be in any suitable form. For example, it can be in the form of a powder, pellet, or a granule (i.e., an aggregate of smaller units of active agent), or small tablets or any combination of any thereof.

The capsule may also include one or more functional excipients such as compressible agent, lubricants, thermal lubricants, antioxidants, binders, diluents, osmotic agents, sweeteners, chelating agents, colorants, flavorants, surfactants, solubilizers, wetting agents, stabilizers, hydrophilic polymers, hydrophobic polymers, waxes, lipophilic materials, absorption enhancers, protease inhibitors, preservatives, absorbents, cross-linking agents, bioadhesive polymers, retardants, and fragrance.

The film forming polymers may be chosen that provide flavoring or taste modifying/masking or moisture barrier include, but not limited to, methylcellulose, hydroxyethyl cellulose, hydroxypropyl cellulose (HPC), hydroxylpropyl methyl cellulose (HPMC) and so on to give a few non-limiting examples.

The film forming polymers may include water soluble polymers comprising, but not limited to, methylcellulose, hydroxyethyl cellulose, hydroxypropyl cellulose (HPC), hydroxylpropyl methyl cellulose (HPMC), and poly(vinylpyrrolidinone) (PVP), polyethylene glycols such as but not limited to PVP, PEG 400, PEG 600, PEG 3350, propylene glycol, polaxamer and povidone, or any combinations of any thereof.

The film forming polymers may include water insoluble polymers comprising, but not limited to, cellulose acetate, ethylcellulose and cellulose derivatives such as cellulose nitrate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, Eudragit® RL, Eudragit® RS, or any combination of any thereof;

The film forming polymers may include pH dependent polymers that is insoluble in aqueous medium at pH lower than 5.5 comprising, but not limited to, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, Eudragit® L30D, Eudragit® L100, Eudragit® FS30D, Eudragit® S I00, Hydroxypropylmethylcellulose Acetate Succinate, or any combinations of any thereof;

The composition of the coating powders may also include pore forming agents, plasticizers, anti-tacky agents, pigments and other additives such as coating powder glidants, or any combinations of any thereof.

Exemplary pore forming agents include water soluble polymers such as methylcellulose, hydroxyethyl cellulose, hydroxypropyl cellulose (HPC), hydroxylpropyl methyl cellulose (HPMC), poly(vinylpyrrolidinone) (PVP), polyethylene glycols such as but not limited to PVP, PEG 400, PEG 600, PEG 3350, propylene glycol, polaxamer and povidone; binders such as lactose, calcium sulfate, calcium phosphate and the like; salts such as sodium chloride, magnesium chloride and the like to give a few examples, and any combinations thereof and other similar or equivalent materials which are widely known in the art.

Plasticizers are used to reduce the glass transition temperature of the coating polymer. Plasticizer can be solid, liquid or plasticizer solution. When the plasticizer is liquid polymers or polymer solutions, it can also be used to decrease the electrical resistivity of the CAPSULE so that the adhesion of coating powder and the coating efficiency could be promoted. Furthermore, liquid plasticizers or plasticizer solutions can provide a strong capillary force between particles and surface of the CAPSULE, enhancing coating powder adhesion. Plasticizers suitable for use in the present invention include, but are not limited to glycerol, propylene glycol, PEG 200-600 grades, triacetin, diethyl phthalate (DEP), dibutyl phthalate (DBP) and tributyl citrate (TBC), triethyl citrate (TEC) and so on.

### EXAMPLE 1

This example illustrates the preparation of capsules of aspirin at 81 mg strength in accordance with the invention. The composition of the formulation is provided in Table 1. The dissolution profile of the powder coated aspirin capsules and the commercial aspirin tablet of the leader brand (Bayer) are presented in FIG. 2.

TABLE 1
Composition of powder coated enteric aspirin capsules

| | | Weight per capsule | |
|---|---|---|---|
| Ingredient | Function | mg | % w/w |

Capsule filling for Example 1 and Example2

| Acetylsalicylic acid (Aspirin) | Active agent | 81.00 | 54.00 |
|---|---|---|---|
| Microcrystalline cellulose | Filler | 68.25 | 45.50 |
| Magnesium stearate | Lubricant | 0.750 | 0.50 |
| Capsule fill weight | | 150.0 | 100.0 |
| Hydroxypropyl methylcellulose | Capsule shell | 36.0 | NA |
| (HPMC) capsule shell (size 4) | | | |

Electrostatic dry powder coating formulation for Example 1

| | | | |
|---|---|---|---|
| Acryl-EZE ® | Enteric coating | 10 g | 80% |
| | polymer mixture | | |
| Polyethylene glycol (PEG 400) | Plasticizer | 2.5 g | 20% |

Weight gain (Coating level) = 8.7%

The method for manufacturing the powder coated aspirin capsules were as follows. Aspirin and microcrystalline cellulose were blended until homogenous. Sieved magnesium stearate was then added to the mixture. The blend was further mixed for 1 minute. The lubricated blend was filled into HPMC capsules. Powder film coating material was milled using suitable milling equipment, such as an air jet mill, to achieve a particle size of less than approximately 20 μm. Filled aspirin capsules were preheated in a non-perforated coating pan to approximately 50° C.

The plasticizer was then sprayed onto the rolling capsules at approximately 0.5 g per minute. The powdered coating material was then deposited onto the capsules at using a corona charging gun at a rate of 1 to 1.5 g per minute at a setting of 40-70 kilo Volts (kV). The plasticizer coating and powdered coating material deposition cycle was repeated until the target coating level was reached.

The coated aspirin-filled HPMC capsules were cured at 50° C. for 60-90 min. The cured capsules were evaluated immediately and after storage under an accelerated stability condition at 40° C. in the presence of desiccants for up to 3 months using the USP dissolution test for delayed release dosage forms.

The USP dissolution test was performed in conditions designed to mimic the environment that is encountered by an oral composition that is swallowed by a human. Although residence time in the stomach varies, the USP test places the composition in the low pH solution of 0.1N HCl at 37±0.5° C. for two hours to mimic the residence time in stomach acid. The composition is then placed in a higher pH aqueous solution, at 6.8±0.05 typically pH 6.8 to mimic the environment of the intestine.

As shown in FIG. 2, the powder coated aspirin capsules exhibit a very similar profile compared to that of the commercial enteric coated aspirin tablets. The negligible release of the active detected during the acid stage indicates that the powder enteric coating of capsule will be effective in providing acid resistance in the stomach upon oral administration. This powder coated aspirin capsule provides satisfactory acid protection with reduced film coating weight. The coating of the powder coated aspirin capsules dissolved rapidly at the buffer stage, suggesting that the capsule shell was effective in preventing an interaction between aspirin or its degradation products and the pH sensitive coating material.

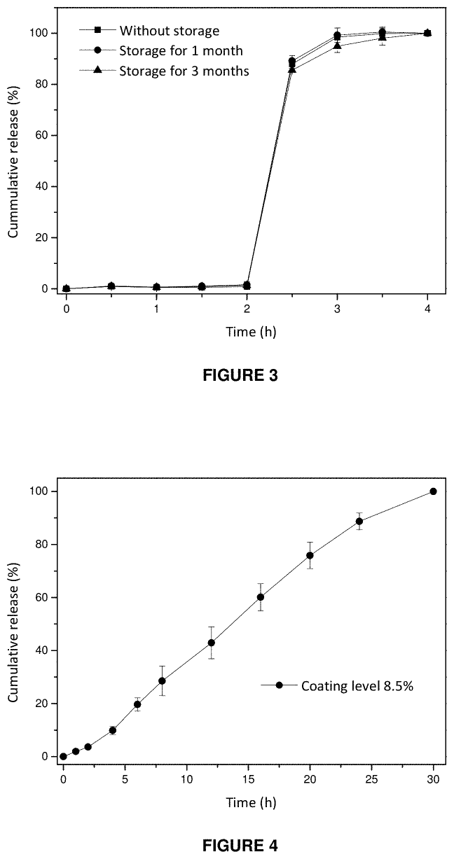

The dissolution profiles of the enteric coated capsules were essentially unchanged after storage under an accelerated stability condition at 40° C. in the presence of desiccants for up to 3 months, as shown in FIG. 3.

### EXAMPLE 2

An extended release capsule of aspirin was prepared using the power coating method presented in this invention using filled uncoated capsules and extended release coating material of the compositions provided in EXAMPLE 1 and Table 2, respectively.

TABLE 2
Electrostatic dry powder coating formulation for Example 2

| Ingredient | Function | weight | % w/w |
|---|---|---|---|
| Eugragit RS/ | Extended coating | 5.6 g | 80 |
| Eudragit RL (1:1) | polymer mixture | | |
| Talc | Anti-tacking agent | 1.34 | 19 |
| Pigment (FD & C Blue #1) | Colorant | 0.03 | 0.5 |
| Colloidal silicone dioxide | Glidant | 0.03 | 0.5 |

| | |
|---|---|
| Coating level | 8.5% |

As shown in FIG. 4, the dissolution profile suggests that the active ingredient can be released for up to 30 hours at a close to zero-order rate.

Pharmaceutical capsules are different than pharmaceutical tablets. Pharmaceutical tablets are solid dosage forms that are formed by APIs and other excipients, compressed into tablet cores and then coated with coating materials to form the tablets. Capsules are containers typically made of HPMC or gelatin, which are filled with APIs and other excipients. Those APIs and/or excipients could be coated or uncoated small particles, granules, pellets or small tablets. Normally capsules are filled with APIs (drug) and other excipients without coating process because the coating process will damage the capsule itself due to the moisture sensitivity of the capsule materials. The present powder coating technology provides a coating method for capsules, making capsules “coat-able”. Powder coating of capsules may provide coatings which protect the capsules from the destruction of the environment and current liquid coating processes (because liquid coating process tends to damage the capsules due to the moisture sensitivity of the capsule materials). The present method of powder coating of capsules are very advantageous in that they avoid contact between the APIs and the outside environment, which is advantageous in those circumstances where the coating environment can damage the APIs.

The powder coating of capsules filling with small uncoated granules (or pellets or small tablets) may save energy and cost compared to the uncoated capsules being filled with coated granules (or pellets or small tablets) because coating of small pellets is typically very complicated and expensive.

The coating membrane may be water soluble to achieve immediate/instant drug release profile, or the coating membrane may be water insoluble with designed permeability to achieve sustained/controlled drug release profile. The material of the coating membrane may be selected to give a membrane which is pH sensitive to achieve enteric coating film and delayed drug release profile.

The coating membrane may be a multilayer that combines water soluble layers and water insoluble layers and also pH sensitive layers, or any two of them, to achieve immediate/instant release and/-or sustained and/-or controlled/-or delayed release. The coating membrane advantageously provides a barrier between the inside APIs (solid or liquid) and the outside environment thereby providing protection for the inside APIs.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

1. A method of producing a dry powder coated soft pharmaceutical capsule, comprising:
preparing a dry powder film forming polymer coating composition, comprised of particles, to be coated onto the outer surface of the soft pharmaceutical capsules, the size of the particles being in a range from about 1 nm to about 500 μm;
placing the soft pharmaceutical capsules into the interior of a rotatable housing of a coater and preheating the soft pharmaceutical capsules;
spraying the dry powder film forming polymer coating composition into the interior to coat the outer surface of the soft pharmaceutical capsules;
rotating the rotatable housing to produce a uniform coating of the dry powder film forming polymer coating composition on the outer surface of the soft pharmaceutical capsules; and
curing the dry coated soft pharmaceutical capsules to form a uniform cured film enveloping each soft pharmaceutical capsule.

2. The method according to claim 1, wherein the capsules are preheated to a temperature close to the glass transition temperature (T_{g}) of the polymer(s) contained in said dry powder film forming polymer coating composition, wherein said polymers are selected to have a glass transition temperature in a range from about 20 to about 200° C.

3. The method according to claim 2, wherein said glass transition temperature is in a range from about 30 to about 100° C.

4. The method according to claim 2, wherein said glass transition temperature is in a range from about 40 to about 60° C.

5. The method according to claim 1, further comprising spraying a suitable amount of plasticizer into the housing to comingle with the dry powder film forming polymer coating composition.

6. The method according to claim 5, wherein said plasticizer is sprayed into the housing prior to spraying the dry powder film forming polymer coating composition.

7. The method according to claim 5, wherein said plasticizer is sprayed into the housing at the same time with spraying the dry powder film forming polymer coating composition.

8. The method according to claim 1, including spraying a suitable amount of plasticizer into said housing during spraying of the dry powder film forming polymer coating composition, said suitable amount of plasticizer being selected to reduce a glass transition temperature (T_{g}) of the dry powder film forming polymer coating composition to a range between about 30 to about 100° C.

9. The method according to claim 8, wherein said plasticizer is any one or combination of a liquid pure plasticizer, a plasticizer in a solution, and a dry powder plasticizer.

10. The method according to claim 1, wherein during curing in the housing the coated capsules are cured at a temperature in a range from about 30 to about 100° C., and wherein the curing time is up to about 4 hours.

11. The method according to claim 10, wherein during curing in the housing the coated capsules are cured at a temperature in a range from about 40 to about 60° C.

12. The method according to claim 1, wherein the dry powder film forming polymer coating composition comprises polymers that form a film for moisture barrier.

13. The method according to claim 1, wherein the dry powder film forming polymer coating composition comprises water soluble polymers that achieve instant or immediate drug release.

14. The method according to claim 1, wherein the dry powder film forming polymer coating composition comprises water insoluble polymers that achieve sustained or controlled drug release.

15. The method according to claim 1, wherein the dry powder film forming polymer coating composition comprises pH dependent polymers that are insoluble in aqueous medium at pH lower than about 5.5, thereby resulting in an enteric coating for delayed drug release.

16. The method according to claim 1, wherein the dry powder film forming polymer coating composition further comprises plasticizers, anti-tacky agents, pore forming agents, or any combination of any thereof.

17. The method according to claim 1, wherein the spraying of the dry powder film forming polymer coating composition is accomplished by electrostatically spraying.

18. The method according to claim 17, wherein the electrostatically spraying is performed using an electrostatic spray gun.

19. The method according to claim 18, wherein the electrostatic spray gun is a corona charging gun or a tribo charging gun.

20. The method according to claim 1, wherein the shell of capsules is made of gelatin, or hydroxylpropyl methyl cellulose (HMPC), or any combination of any thereof.

21. The method according to claim 1, wherein at least one pharmaceutically active agent contained in the soft pharmaceutical capsule is in the form of semi-solids or liquids.

22. The method according to claim 1, wherein the film forming polymers include water soluble polymers comprising, methylcellulose, hydroxyethyl cellulose, hydroxypropyl cellulose (HPC), hydroxylpropyl methyl cellulose (HPMC), poly(vinylpyrrolidinone) (PVP), and polyethylene glycols comprising PVP, PEG 400, PEG 600, PEG 3350, propylene glycol, polaxamer and povidone, or any other water soluble polymers, or any combination of any thereof.

23. The method according to claim 1, wherein the film forming polymers include water insoluble polymers comprising
2-methylprop-2-enoic acid-n,n-dimethylmethanamine(2:1),
ethyl prop-2-enoate; methyl 2-methylprop-2-enoate;
trimethyl-[2-(2-methylprop-2-enoyloxy)ethyl]azanium; chloride, cellulose acetate, ethylcellulose, cellulose derivatives, cellulose nitrate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, or any combination of any thereof.

24. The method according to claim 1, wherein the film forming polymers include pH dependent polymers that is insoluble in aqueous medium at pH lower than 5.5 comprising, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, poly(ethyl acrylate-co-methyl methacrylate-co -trimethylammonioethyl methacrylate chloride) 1:2:0.2, poly(methacylic acid-co-methyl methacrylate) 1:1, poly(methyl acrylate-co-methyl methacrylate-co-methacrylic acid) 7:3:1, and poly(methacylic acid-co-methyl methacrylate) 1:2, Hydroxypropylmethylcellulose Acetate Succinate or any combinations of any thereof.

25. The method according to claim 1, further comprising producing one or more orifices through the uniform cured film to expose the outer surface of the capsules at the position of each of the one or more orifices, and wherein the one or more orifices are produced by using any one of mechanical drilling, laser drilling, or an indentation method for forming osmotic capsules.

26. The method according to claim 1, wherein the coater is a pan coater.

27. A pharmaceutical capsule having a dry powder polymer film coating produced using the method of claim 1.
